(19) United States
(12) Patent Application Publication
(10) Pub. No.: US 2013/0237901 A1
(43) Pub. Date: Sep. 12, 2013

(54) APPARATUS AND METHODS TO TREAT OR PREVENT KIDNEY AND UROLOGIC DISEASE

(71) Applicant: Sang Hoon Woo, Palo Alto, CA (US)

(72) Inventor: Sang Hoon Woo, Palo Alto, CA (US)

(73) Assignee: THE BOARD OF TRUSTEES OF THE LELAND STANFORD JUNIOR UNIVERSITY, Palo Alto, CA (US)

(21) Appl. No.: 13/807,919

(22) PCT Filed: Jun. 30, 2011
(86) PCT No.: PCT/US11/42717

§ 371 Date: May 21, 2013

Related U.S. Application Data
(60) Provisional application No. 61/398,810, filed on Jul. 1, 2010.

(51) Int. Cl. A61M 13/00; A61F 7/00

(52) U.S. Cl. CPC A61M 13/003; A61F 7/0085
USPC 604/24; 604/26; 607/105

(57) ABSTRACT

An apparatus for treating or preventing a kidney disease includes a delivery device configured for insertion into an urinary tract, wherein the delivery device comprises a section for placement in the urinary tract, and wherein the delivery device comprises a first end configured for coupling to an oxygen source, and a lumen configured for delivering oxygen from the oxygen source to a kidney.

### RELATED APPLICATION DATA

This application is the national phase of PCT Application No. PCT/US2011/042717 filed on Jun. 30, 2011, which claims priority to and the benefit of U.S. Provisional Patent Application No. 61/398,810, filed on Jul. 1, 2010, the entire disclosures of both of which are expressly incorporated by reference herein.

### PRIORITY DATA

This application claims priority to and the benefit of U.S. Provisional Patent Application No. 61/398,810, filed on Jul. 1, 2010, the entire disclosure of which is expressly incorporated by reference herein.

### FIELD

This application relates generally to systems and methods for treating or preventing kidney and urologic diseases.

### BACKGROUND

Kidney disease is a major public health problem. Acute kidney injury (acute renal failure) in particular, occurs commonly in a hospital setting and it is associated with high mortality and other life threatening complications. When a kidney fails, it could cause dangerous electrolyte abnormalities, seizure, respiratory failure, and fluid overload. Treatment has been limited for this serous medical problem. IV fluid therapy has been used with limited benefit. When acute kidney injury progresses, a patient with this condition may require dialysis for short term or long term.

Thus, Applicant of the subject application determines that new systems and methods for treating or preventing kidney disease would be desirable.

### SUMMARY

In accordance with some embodiments, an apparatus for treating or preventing a kidney disease includes a delivery device configured for insertion into an urinary tract, wherein the delivery device comprises a section for placement in the urinary tract, and wherein the delivery device comprises a first end configured for coupling to an oxygen source, and a lumen configured for delivering oxygen from the oxygen source to a kidney.

In one or more embodiments, the apparatus may include a controller configured for controlling oxygen flow from the oxygen source.

In one or more embodiments, the apparatus may include a sensor for sensing a characteristic representative of an oxygen level, wherein the controller is configured for controlling the oxygen flow based at least in part on the sensed characteristic.

In one or more embodiments, the controller may be configured for controlling the oxygen flow based at least in part on the sensed characteristic to maintain a target oxygen level in urine.

In one or more embodiments, the controller may be configured for controlling a flowing rate of oxygen through the delivery device.

In one or more embodiments, the controller may include a user interface configured for receiving an input from a user to control a rate of oxygen flowing through the delivery device.

In one or more embodiments, the user interface may be configured for receiving an oxygen flow rate as the input.

In one or more embodiments, the controller may be configured to deliver oxygen continuously.

In one or more embodiments, the controller may be configured to deliver oxygen intermittently.

In one or more embodiments, the apparatus may include an urinary catheter for draining urine from the kidney.

In one or more embodiments, the apparatus may include an antibiotic medication for delivery by the delivery device.

In one or more embodiments, the apparatus may include the oxygen source, wherein the oxygen source comprises fluid that is supersaturated with oxygen.

In one or more embodiments, the apparatus may include a source of cooling material, wherein the delivery device is coupled to the source of cooling material, and is configured to deliver the cooling material to the kidney.

In one or more embodiments, the cooling material may include nitrous oxide, carbon dioxide, cooled fluid, liquid nitrogen, or argon gas.

In one or more embodiments, the delivery device may include an additional lumen for delivery of the cooling material.

In one or more embodiments, the delivery device may include a catheter.

In accordance with other embodiments, a method for treating or preventing a kidney disease includes inserting a delivery device into an urinary tract, advancing the delivery device distally inside the urinary tract until a distal end of the delivery device is at a desired position, and delivering oxygen through the delivery device in the urinary tract to a kidney.

In one or more embodiments, the act of delivering oxygen may include delivering oxygenated fluid.

In one or more embodiments, the oxygenated fluid may be supersaturated with oxygen.

In one or more embodiments, the oxygenated fluid may be delivered at a rate sufficient to reduce a kidney injury from a disease or from a surgery.

In one or more embodiments, the disease may be sepsis, heart failure, infection, kidney inflammation, hypotension, or acute kidney failure.

In one or more embodiments, the method may include delivering a cooling material through the urinary tract to cool the kidney.

In one or more embodiments, the kidney may be cooled to prevent acute kidney injury.

In one or more embodiments, the kidney may be cooled to inhibit inflammation of the kidney.

In one or more embodiments, the kidney may be cooled to a temperature that is anywhere from 37 degree C. and −50 degree C.

In one or more embodiments, the kidney may be cooled for a duration that is anywhere from 0.01 second to 8 weeks.

In one or more embodiments, the kidney may be cooled continuously.

In one or more embodiments, the kidney may be cooled intermittently.

In accordance with other embodiments, an apparatus for treating or preventing a kidney disease includes a delivery device configured for insertion into an urinary tract, wherein the delivery device comprises a section for placement in the urinary tract, and wherein the delivery device comprises a first end configured for coupling to a source of cooling material, and a lumen configured for delivering the cooling material to a kidney.

In one or more embodiments, the cooling material may include nitrous oxide, carbon dioxide, cooled fluid, liquid nitrogen, or argon gas.

In one or more embodiments, the delivery device may also be configured for coupling to an oxygen source for delivering oxygen through the urinary tract to the kidney.

In one or more embodiments, the delivery device may include an additional lumen for delivery of the oxygen to the kidney.

In one or more embodiments, the apparatus may include a controller configured for controlling flow of the cooling material.

In one or more embodiments, the apparatus may include a sensor for sensing a characteristic representative of a temperature, wherein the controller is configured for controlling the flow of the cooling material based at least in part on the sensed characteristic.

In one or more embodiments, the controller may be configured for controlling the flow based at least in part on the sensed characteristic to maintain a target temperature.

In one or more embodiments, the controller may be configured for controlling a flowing rate of the cooling material through the delivery device.

In one or more embodiments, the controller may include a user interface configured for receiving an input from a user to control the flow of the cooling material.

In one or more embodiments, the user interface may be configured for receiving a temperature value as the input.

In one or more embodiments, the apparatus may include an urinary catheter for draining urine from the kidney.

In accordance with other embodiments, a method for treating or preventing a kidney disease includes inserting a delivery device into an urinary tract, advancing the delivery device distally inside the urinary tract until a distal end of the delivery device is at a desired position, and delivering cooling material through the delivery device in the urinary tract to a kidney.

In one or more embodiments, the kidney may be cooled to prevent acute kidney injury.

In one or more embodiments, the kidney may be cooled to inhibit inflammation of the kidney.

In one or more embodiments, the kidney may be cooled to a temperature that is anywhere from 37 degree C. and −50 degree C.

In one or more embodiments, the kidney may be cooled for a duration that is anywhere from 0.01 second to 8 weeks.

Other and further aspects and features will be evident from reading the following detailed description of the embodiments.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates an apparatus for treating or preventing a kidney disease in accordance with some embodiments.

FIG. 2 illustrates a method of using the apparatus of FIG. 1 in accordance with some embodiments.

FIG. 3 illustrates another method of using the apparatus of FIG. 1 in accordance with other embodiments.

FIG. 4 illustrates another apparatus for treating or preventing a kidney disease in accordance with other embodiments.

FIG. 5 illustrates another apparatus for treating or preventing a kidney disease in accordance with other embodiments.

FIG. 6 illustrates another apparatus for treating or preventing a kidney disease in accordance with other embodiments.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

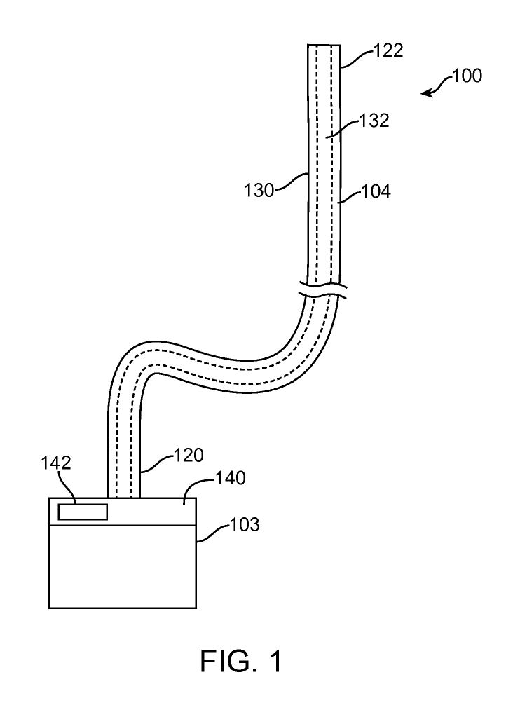

FIG. 1 illustrates an apparatus 100 for treating or preventing kidney disease in accordance with some embodiments. The apparatus 100 includes a delivery device 104 that is coupled to an oxygen source 103. The device 104 includes a proximal end 120 configured (e.g., sized and/or shaped) to detachably couple to the oxygen source 103, and a distal end 122 configured for placement into a patient body through an urinary tract. The device 104 also includes at least a section 130 configured (e.g., sized and/or shaped) for placement into an urinary tract of the patient, and a lumen 132 for delivering oxygen from the oxygen source 103 through an urinary tract to a kidney. In some embodiments, the device 104 has a cross-sectional dimension that is anywhere from 0.03 cm to 3 cm, and more preferably, anywhere from 0.05 cm to 0.5 cm. This allows the device 104 to be inserted into the urinary tract and be advanced distally within the urinary tract. Also, as illustrated in the figure, the distal end 122 of the device 104 has a blunt tip. This allows the distal end 122 of the device 104 to be inserted and advanced within the urinary tract without causing tissue damage at an interior wall of the urinary tract. In some embodiments, the delivery device 104 may be implemented as a catheter configured for insertion into an urinary tract.

In the illustrated embodiments, the oxygen source 103 includes a container containing fluid that has oxygen (e.g., liquid or gas that is saturated or supersaturated with oxygen). In one example, the fluid may be saline that includes oxygen. The oxygen source 103 is in fluid communication with the lumen 132 of the device 104 so that the fluid from the oxygen source 103 may be delivered to the distal end 122 of the device 104 through the lumen 132. In other embodiments, instead of delivering oxygen using fluid that is saturated with oxygen, the oxygen source 103 may contain oxygen that is not saturated in any fluid. In such cases, the oxygen source 103 may be configured to deliver the oxygen in the form of oxygen molecule or compound directly. In further embodiments, the fluid in the oxygen source 103 may optionally also include an antibiotic medication, antifungal medication, and/or another drug, for treating a medical condition in the patient. Use of antibiotic medication and/or antifungal medication in combination with the oxygen delivery is advantageous because it may prevent or treat infection along the urinary tract and/or at the kidney(s).

In the illustrated embodiments, the apparatus 100 further includes a controller 140 for controlling a flow (e.g., flow rate or amount) of the fluid containing oxygen from the oxygen source 103. The controller 140 may be configured (e.g., programmed and/or built) to deliver oxygen continuously or intermittently. In some embodiments, the rate of oxygen flow ranges between 1 ml per minute and 5 liter per minute. In other embodiments, the oxygen may be delivered to a kidney for five minutes and then the delivery stops for five minutes, and then oxygen is delivered again for five minutes according to predetermined protocol. In further embodiments, oxygen may be delivered for 1 hour, and then the controller 140 stops delivering oxygen for 30 min. In other embodiments, the duration of oxygen delivery may be different from the above examples, and the duration of no oxygen delivery may be different from the above examples. In further embodiments, other intermittent delivery protocols may be used.

Also, in some embodiments, the apparatus 100 may optionally further include a sensor for sensing a characteristic representative of an oxygen level in the urine. In such case, the controller 140 may be configured (e.g., programmed and/or built) to control the oxygen flow based at least in part on the sensed characteristic, so that a target oxygen level in the urine is maintained. The sensor may be located at the distal end 122, and the apparatus 100 may include one or more wires coupling the sensor to the controller 140. The wire(s) may be housed within a wall of the delivery device 104, or within the lumen 132. During use, the wire(s) transmit signal(s) from the sensor to the controller 140, which processes the signal(s) (e.g., comparing the signal with a prescribed value) and controls the flow of oxygen from the source 103 based on the processing of the signal(s). In one implementation, the sensor is configured to sense an amount of oxygen in the urine. In such cases, the controller 140 is configured to compare the sensed signal from the sensor with a prescribe value (which represents a desired amount of oxygen), and control a flow of the oxygen from the source 103. For example, if the sensed oxygen level is below the prescribed value, the controller 140 then increases the flow of oxygen, and vice versa. In other embodiments, the sensor may be located in other locations at the apparatus 100. For examples, the sensor may be located at the proximal end of the device 104, in the middle of the device 104, anywhere along the length of the device 104, inside an urine collection device, inside an urine collection bag, or anywhere within an urinary tract.

In the illustrated embodiments, the controller 140 also includes an interface 142 that allows a user to operate (e.g., program, control, etc.) the controller 140. In one implementation, the interface 142 (which may include keys, buttons, touchscreen, touchpad, trackball, dial knob, etc.) is configured to receive an input from a user for controlling a rate or an amount of oxygen flowing through the delivery device 104. For example, the input may be an oxygen flow rate, or an oxygen amount, that is input by the user. In such cases, based on the prescribed oxygen flow rate/amount, the controller 140 then causes oxygen to be delivered from the oxygen source 103 (e.g., by operating a pump and/or a valve) so that the prescribed oxygen flow rate/amount is achieved. In another example, the input may be a targeted oxygen level in urine. In such cases, based on the prescribed oxygen level, the controller 140 then causes oxygen to be delivered from the oxygen source 103 (e.g., by operating a pump and/or a valve) so that the targeted oxygen level in the urine is achieved.

In other embodiments, the user interface 412 may be configured for allowing a user to input oxygen flow rate, delivered oxygen amount, mode of oxygen delivery (e.g., continuously or intermittently), and oxygen delivery protocols. Based on the operation of the user interface 412 by a user, the controller 140 then controls a rate of oxygen flow, an amount of oxygen flow, and/or manner of oxygen flow (e.g., delivering oxygen continuously, intermittently, or based on selected protocol). In one example, the oxygen delivery protocol may be delivery based on measured oxygen level in urine. In such cases, the controller 140 adjusts the rate of oxygen infusion based on a measured oxygen level of urine from a kidney to maintain a target oxygen level in the urine. Example of urine oxygen level is urine oxygen tension.

In any of the embodiments described herein, the apparatus 100 may optionally further include an additional lumen for draining urine from the kidney. In some embodiments, the additional lumen may be in the delivery device 104 (e.g., next to the lumen 132 in a side-by-side configuration). In other embodiments, the additional lumen may be a part of an urinary catheter that is different from the device 104. During use, the additional lumen is configured to drain urine from the kidney, as oxygen containing fluid is being supplied to the kidney from the oxygen source 103.

Also, in any of the embodiments described herein, the delivery device 104 may optionally further include an anchoring device to maintain the location of the device 104 within the urinary tract. For example, in some embodiments, the delivery device 104 may include a ballon at the distal end, or anywhere along the length of the device 104. During use, the balloon may be expanded to contact surrounding tissue in the urinary tract, thereby securing the device 104 relative to the urinary tract.

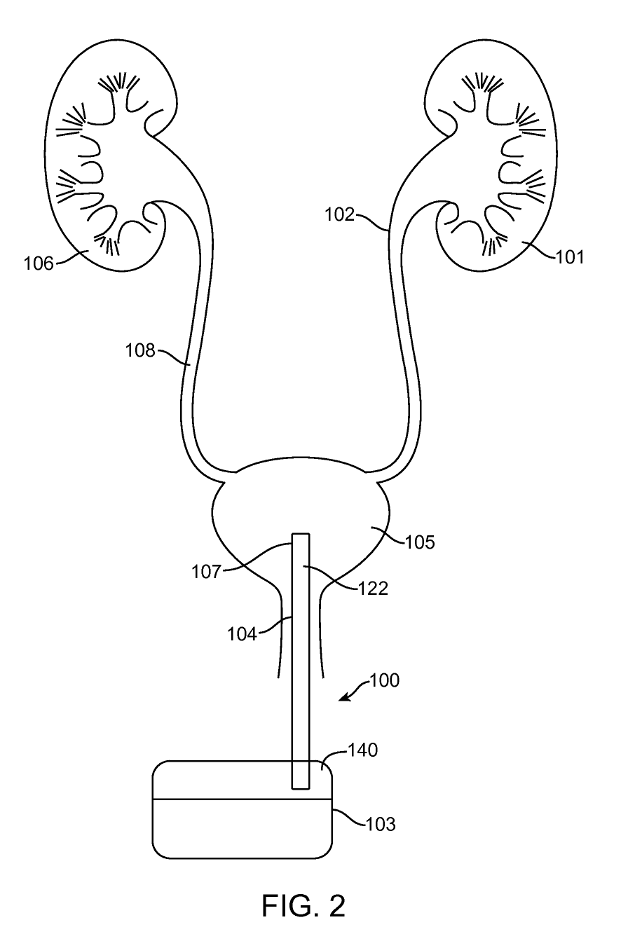

FIG. 2 illustrates a method of using the apparatus 100 of FIG. 1 in accordance with some embodiments. First, the delivery device 104 is connected to the oxygen source 103. The distal end 122 (with the distal tip 107) of the delivery device 104 is then inserted into an urinary tract, and is advanced distally within the urinary tract until the distal end 122 is placed at a desired position. As shown in the figure, the urinary tract includes a urethra, bladder 105, ureter 102, renal calyx, and renal pelvis. In the illustrated embodiments, the tip 107 of the delivery device 104 is placed inside the bladder 105 of the urinary tract. The controller 140 is then operated (e.g., to control a pump) to deliver oxygen from the oxygen source 103 through the urinary tract to the kidneys 101, 106. In particular, the oxygen is delivered from the oxygen source 103 through the delivery device 104 that is located in the urinary tract, and exits the distal end 122 of the delivery device 104 at the bladder 105. The oxygen delivered at the bladder 105 then passes through the ureters 102, 108 to kidneys 101, 106, respectively. Thus, as used in this specification, the phrase “delivering oxygen from the oxygen source to a kidney”, or similar phrases, may refer to any manner in which the oxygen is transmitted from the oxygen source to the kidney (e.g., directly or indirectly), and does not necessarily require any delivery device to be placed at a kidney (although in some embodiments described herein, the tip 107 of the delivery device 104 may be placed at a kidney).

In some embodiments, the method for delivering oxygen to the kidney involves infusing oxygen from the oxygen source 103. In other embodiments, if the oxygen source 103 contains oxygenated fluid (e.g., saline), the method for delivering oxygen to the kidney may involve delivering oxygenated fluid (e.g., in an amount of 10-1500 ml) from the oxygen source 103 through the urinary tract to the kidney. In particular, the oxygen-containing fluid may be delivered to one or both kidneys through the device 104 located in the urinary tract. In the illustrated embodiments, the tip 107 of the delivery device 104 is located within a bladder. In such cases, once the oxygen-containing fluid is delivered out of the lumen 132 at the bladder, the oxygen-containing fluid will pass the bladder (e.g., when the bladder is filled), and then a ureter(s), and then to a kidney (or both kidneys). The oxygen containing fluid may remain in the kidney(s) for a certain period of time. The oxygen containing fluid may remain in the kidney(s) for a period that is anywhere between 1 second and 10 minutes in an implementation until the oxygen containing fluid is drained out. In another implementation, the period may last less than 1 second, or last longer than 10 minutes. In some embodiments, the controller 140 is configured to deliver oxygen from the oxygen source 103 at a rate sufficient to reduce or prevent a kidney injury from a disease, which may include sepsis, heart failure, infection, kidney inflammation, hypotension, and/or acute kidney failure, or from a surgery.

In other embodiments, the fluid delivered to the kidney may be supersaturated with oxygen. In one implementation, oxygen is pressurized and dissolved into saline. This saline is saturated or supersaturated with oxygen containing higher amount of oxygen. In some embodiments, the oxygen concentration in the saline may be adjusted by a controller. In other embodiments, fluid with a fixed amount of dissolved oxygen may be used. The oxygen concentration may be anywhere from 10 mmHg to 1,000 mmHg in some embodiments. The oxygen concentration may be less than 10 mmHg, or greater than 1,000 mmHg in other embodiments.

In some embodiments, if the delivery device 104 includes an additional lumen for draining urine, or if an additional urine catheter is provided, then the additional lumen or the urine catheter may be used to drain urine when oxygen is being delivered from the oxygen source 103. In some embodiments, the apparatus 100 may be configured to repeat the acts of delivering oxygen containing fluid to the kidney(s), and draining out the fluid.

Also, in some embodiments in which the delivery device 104 includes a sensor at its distal end 122, the sensor may be used to sense a characteristic that represents an oxygen level in the urine. In such cases, the sensor transmits a signal that represents the oxygen level to the controller 140. The controller 140 compares the sensed oxygen level to a desired oxygen level, and adjusts a pump and/or valve at the oxygen source 103 to control an amount of oxygen being delivered to the kidney 101. For example, in some embodiments, the controller 140 may operate the pump to pump more or less oxygenated fluid into the patient body. In other embodiments, the controller 140 may partially or completely close a valve to reduce or prevent additional oxygenated fluid from being delivered into the patient body. In further embodiments, the controller 140 may increase oxygen saturation in the fluid by advancing a piston to increase hydrostatic pressure.

As illustrated in the above embodiments, delivering oxygen to the kidney is advantageous because it may reduce or prevent a kidney injury from a disease, which may include sepsis, heart failure, infection, kidney inflammation, hypotension, acute kidney failure, and/or dehydration. Delivering oxygen to the kidney(s) is also advantageous because it may prevent or treat acute kidney injury, acute renal failure, acute kidney injury on chronic kidney disease, or chronic kidney disease. Acute kidney injury or acute renal failure occurs frequently in the setting of infection, hypotension, dehydration, shock, sepsis, kidney inflammation, glomerulonephritis, surgery, or drug side effect which may lead to low oxygen state in a kidney. Delivering oxygen to a kidney may help prevent or reduce deterioration of a kidney function, thereby preventing or treating kidney injury. Also, delivering oxygen using the urinary tract is advantageous because it is readily accessible, and does not require open surgery on the patient. Furthermore, placement of the tip of the delivery device 104 at the bladder is advantageous because it does not require the delivery device 104 to be placed distal to the bladder, and obviates the need to do extra work to steer the delivery device 104 through the ureter 102/108.

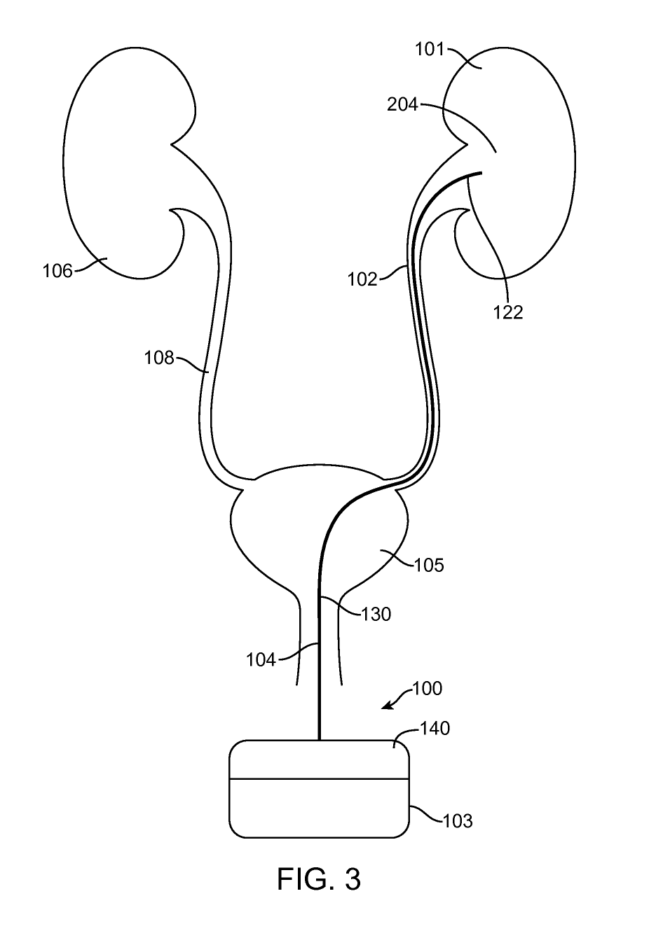

In other embodiments, the tip 107 of the delivery device 104 may be placed within other areas, such as at other parts of the urinary tract (e.g., at the urethra, the ureter, the renal pelvis, the renal calyx), or at the kidney(s). In such cases, the delivery device 104 may be deployed twice to delivery oxygen two times from the oxygen source 103, once for the right kidney, and again for the left kidney. FIG. 3 illustrates another method of using the apparatus 100 in accordance with other embodiments, particularly showing the tip 207 of the delivery device 104 being placed in the renal pelvis 204 to deliver oxygen from oxygen source 103 to the kidney 205.

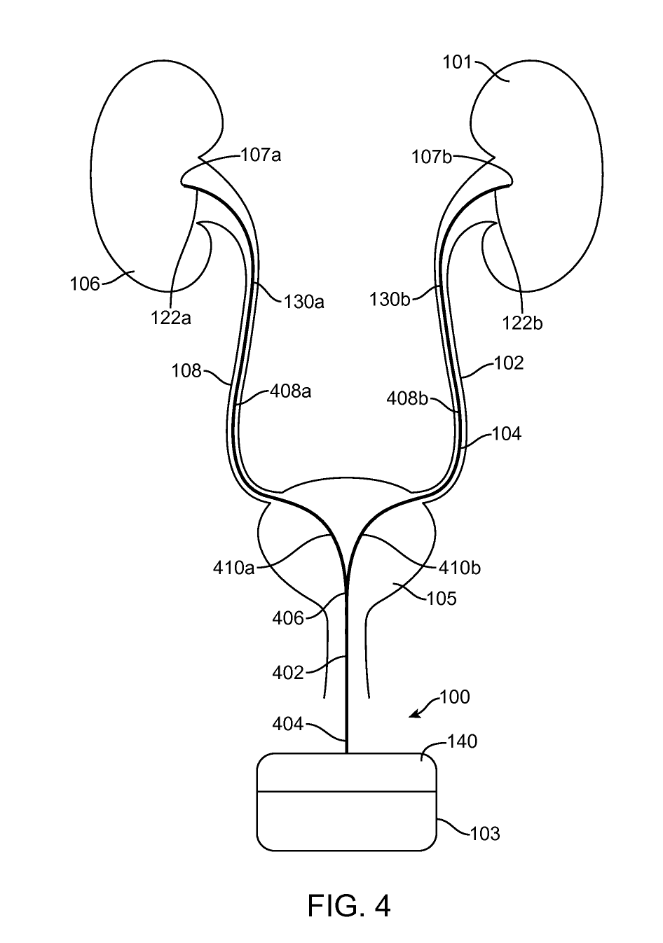

FIG. 4 illustrates another variation of the apparatus 100 in accordance with other embodiments. The apparatus 100 is similar to the apparatus 100 of FIG. 1, except that the delivery device 104 has two distal ends 122 instead of one. In particular, as shown in the illustrated embodiments, the delivery device 104 has a proximal segment 402 with a proximal end 404 that is coupled to the oxygen source 103, and a distal end 406 that is coupled with a proximal end 410a of a first distal segment 408a, and with a proximal end 410b of a second distal segment 408b. The first distal segment 408a includes a distal end 122a, and the second distal segment 408b includes a distal end 122b. The first and second distal segments 408a, 408b also includes respective sections 130a, 130b for placement into the urinary tracts, and respective lumens (not shown) that are in fluid communication with the proximal segment 402 and the oxygen source 103. During use, the first and second distal segments 408a, 408b are placed in the ureters with their respective tips 107a, 107b being placed at the renal pelvis 204 to deliver oxygen from the oxygen source 103 to the respective kidneys. In other embodiments, the tips 107a, 107b may be placed in the respective ureters, or at the respective kidneys 101, 106.

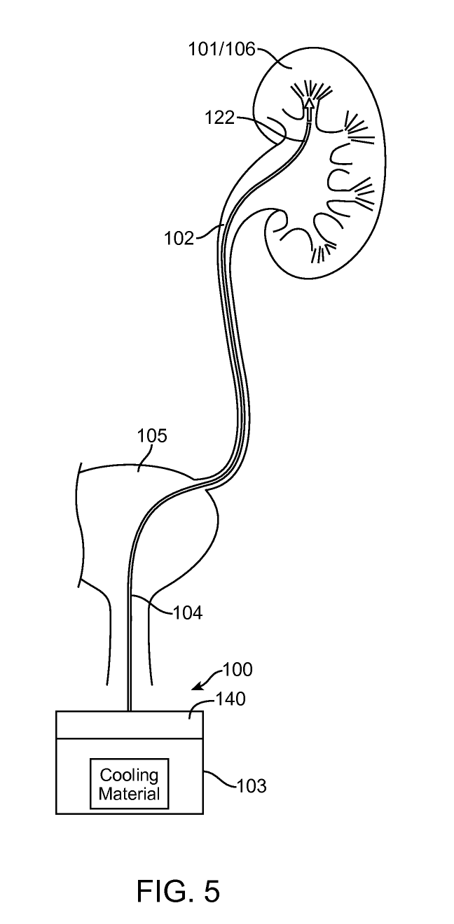

FIG. 5 illustrates another apparatus 100 in accordance with other embodiments. The apparatus 100 of FIG. 5 is similar to the apparatus 100 of FIG. 1 (and may have any or all of the features of the apparatus 100 of FIG. 1), except that the source 103 contains cooling material instead of oxygen. Suitable cooling material may include, but not limited to liquid nitrous oxide, nitrous oxide gas, liquid nitrogen, argon gas, liquid carbon dioxide, solid carbon dioxide, carbon dioxide gas, cooled liquid (e.g., cooled saline, cooled water, etc.), and the like.

When using the apparatus of FIG. 5, first, the delivery device 104 is connected to the cooling material source 103. The distal end 122 (with the distal tip 107) of the delivery device 104 is then inserted into an urinary tract, and is advanced distally within the urinary tract until the distal end 122 is placed at a desired position. As shown in the figure, the urinary tract includes a urethra, bladder 105, ureter 102, renal calyx, and renal pelvis. In the illustrated embodiments, the tip 107 of the delivery device 104 is placed inside the bladder 105 of the urinary tract. The controller 140 is then operated (e.g., to control a pump) to deliver cooling material from the cooling material source 103 through the urinary tract to the kidneys 101, 106. In particular, the cooling material is delivered from the source 103 through the delivery device 104 that is located in the urinary tract, and exits the distal end 122 of the delivery device 104 at the bladder 105. The cooling material delivered at the bladder 105 then passes through the ureters 102, 108 to kidneys 101, 106, respectively. Thus, as used in this specification, the phrase “delivering cooling material to a kidney”, or similar phrases, may refer to any manner in which the cooling material is transmitted from the cooling material source to the kidney (e.g., directly or indirectly), and does not necessarily require any delivery device to be placed at a kidney (although in some embodiments described herein, the tip 107 of the delivery device 104 may be placed at a kidney).

In the illustrated embodiments, the tip 107 of the delivery device 104 is located within a bladder. In such cases, once the cooling material is delivered out of the lumen 132 at the bladder, the cooling material will pass the bladder (e.g., when the bladder is filled), and then a ureter(s), and then to a kidney (or both kidneys). The cooling material may remain in the kidney(s) for a certain period of time. In some embodiments, the controller 140 is configured to deliver cooling material from the source 103 at a rate sufficient or amount to cool the kidney(s) to thereby reduce or prevent the kidney injury from a disease, which may include sepsis, heart failure, infection, kidney inflammation, hypotension, and/or acute kidney failure. In some embodiments, the cooling material is delivered to the kidney(s) to lower the temperature of a kidney to a range between about 37 degree C. and −50 degree C. Also, in some embodiments, the cooling time may range from 0.01 second to 8 weeks. In other implementations, the cooling time may be less than 0.01 second or greater than 8 weeks.

In some embodiments, if the delivery device 104 includes an additional lumen for draining urine, or if an additional urine catheter is provided, then the additional lumen or the urine catheter may be used to drain urine when cooling material is being delivered from the source 103. In some embodiments, the apparatus 100 may be configured to repeat the acts of delivering cooling fluid to the kidney(s), and draining out the fluid.

Also, in some embodiments in which the delivery device 104 includes a sensor at its distal end 122, the sensor may be used to sense a characteristic that represents temperature (e.g., temperature of the urine, kidney, etc.). In such cases, the sensor transmits a signal that represents the temperature to the controller 140. The controller 140 compares the temperature level to a desired temperature level (which may be input into the controller 140 using the interface 142), and adjusts a pump and/or valve at the cooling material source 103 to control an amount of cooling material being delivered to the kidney 101. For example, in some embodiments, the controller 140 may operate the pump to pump more or less cooling material into the patient body. In other embodiments, the controller 140 may partially or completely close a valve to reduce or prevent additional cooling material from being delivered into the patient body. In other embodiments, the sensor sensing a characteristic that represents temperature may be located in anywhere within the urinary tract, in a urine collection device, in a urine collection bag, or in any location at the cooling material delivery device.

As illustrated in the above embodiments, delivering cooling material to the kidney to cool the kidney is advantageous because it may reduce or prevent a kidney injury from a disease, which may include sepsis, heart failure, infection, kidney inflammation, hypotension, acute kidney failure, and/or dehydration. Delivering cooling material to the kidney(s) is also advantageous because it may prevent or treat acute kidney injury, acute renal failure, acute kidney injury on chronic kidney disease, or chronic kidney disease. Embodiments of the kidney cooling device and methods may inhibit deterioration of a kidney function, prevent acute kidney injury, and/or inhibit inflammation of a kidney. The lowered temperature at the kidney may reduce metabolism of kidney. Thus, the lowering temperature of one or both kidneys may have protective effect on the kidney in the setting of acute kidney injury, acute renal failure, shock, sepsis, hypotension, acute inflammation. Also, delivering cooling material using the urinary tract is advantageous because it is readily accessible, and does not require open surgery on the patient. Furthermore, placement of the tip of the delivery device 104 at the bladder is advantageous because it does not require the delivery device 104 to be placed distal to the bladder, and obviates the need to do extra work to steer the delivery device 104 through the ureter 102/108.

In other embodiments, the tip 107 of the delivery device 104 may be placed within other areas, such as at other parts of the urinary tract (e.g., at the urethra, the ureter, the renal pelvis, the renal calyx), or at the kidney(s). In such cases, the delivery device 104 may be deployed twice to delivery cooling material two times from the source 103, once for the right kidney, and again for the left kidney. In other embodiments, the tip 207 of the delivery device 104 may be placed in the renal pelvis 204 to deliver cooling material from source 103 to the kidney 205. In further embodiments, the delivery device 104 may be placed adjacent to an outer surface of a kidney (e.g., by inserting the device 104 through a patient's skin), for delivering the cooling material from the reservoir to the cooling device to lower the temperature of the kidney.

In other embodiments, a kidney treatment system may include an oxygen delivery apparatus that includes an oxygen source for holding oxygen, a delivery device connected to the oxygen source and located within a urinary tract, for delivering oxygen from the oxygen source through a urinary tract to kidney(s), and a kidney cooling device that includes a cooling material source for holding a cooling material, a delivery device connected to the cooling material source and located within the urinary tract, for delivering the cooling material from the cooling material source to kidney(s). In some embodiments, the delivery device for delivering oxygen and the delivery device for delivering the cooling material may be separate devices (e.g., two catheters with two respective lumens), or may be implemented using the same device (e.g., a catheter with two lumens).

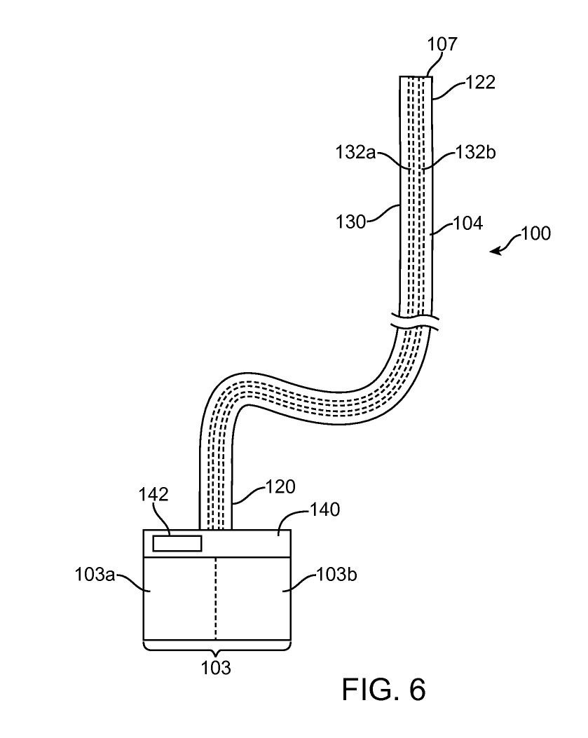

FIG. 6 illustrates another apparatus 100 for treating or preventing kidney disease in accordance with other embodiments. The apparatus 100 includes a delivery device 104 having a proximal end 120 coupled to fluid source 103, and a distal end 122. In the illustrated embodiments, the source 103 includes an oxygen source 103a, and a cooling material source 103b. The delivery device 104 includes a first lumen 132a in fluid communication with the oxygen source 103a for delivering oxygen, and a second lumen 132b in fluid communication with the cooling material source 103b for delivering cooling material. The apparatus 100 also includes a controller 140 with a user interface 142 for controlling flow of oxygen and cooling material from the oxygen source 103a and cooling material source 103b, respectively. In some embodiments, the user interface 142 may have controls that are like those described with reference to the oxygen delivery device of FIG. 1, and controls that are like those described with reference to the cooling material delivery device of FIG. 5.

During use, the distal tip 107 of the delivery device 104 is inserted into the urinary tract, and is advanced distally. The tip 107 may be placed anywhere along the urinary tract, or at the kidney. The controller 140 then delivers oxygen from the oxygen source 103a, and cooling material from the cooling material source 103b, through the urinary tract to reach the kidney. In some embodiments, the tip 107 may be placed at the bladder, and the oxygen and cooling material are delivered to the bladder. The delivered oxygen and cooling material then travels from the bladder to reach the kidney(s). In other embodiments, the tip 107 may be placed distal to the bladder. For example, the tip 107 may be placed at the kidney, in which cases, the oxygen and the cooling material will be delivered out of the distal tip 107 and directly to the kidney.

As illustrated in the above embodiments, delivering both oxygen and cooling material to the kidney is advantageous because it may effectively reduce or prevent a kidney injury from a disease, which may include sepsis, heart failure, infection, kidney inflammation, hypotension, acute kidney failure, and/or dehydration, or from a surgery. Delivering both oxygen and cooling material to the kidney(s) is also advantageous because it may effectively prevent or treat acute kidney injury, acute renal failure, acute kidney injury on chronic kidney disease, or chronic kidney disease.

In other embodiments, the apparatus 100 may include two distal segments for delivering oxygen and cooling material to both kidneys (e.g., simultaneously), like that described with reference to the embodiments of FIG. 4. Also, in other embodiments, instead of having one controller 140 for controlling both the flow of oxygen and cooling material, the apparatus 100 may include separate controllers 140 for controlling flow of oxygen and cooling material, respectively. In addition, in other embodiments, instead of having a source 103 that contains both the oxygen and the cooling material, the oxygen source 103a and the cooling material source 103b may be separate from each other. Also, in other embodiments, the delivery device 104 in the apparatus 100 of FIG. 6 may include a third lumen for draining urine. In further embodiments, the apparatus 100 may include a sensor for sensing a characteristic representative of oxygen level in urine, and/or a sensor for sensing a characteristic representative of temperature (e.g., of urine or kidney). In such cases, the controller 140 may be configured to control flow of oxygen and/or cooling material based at least in part on the sensed characteristic(s).

In other embodiments, instead of using the apparatus 100 of FIG. 6, the oxygen delivery apparatus 100 of FIG. 1, and the cooling material delivery apparatus 100 of FIG. 5, may be used together to deliver oxygen and cooling material to the kidney(s).

Although particular embodiments have been shown and described, it will be understood that they are not intended to limit the claimed inventions, and it will be obvious to those skilled in the art that various changes and modifications may be made without departing from the spirit and scope of the claimed inventions. The specification and drawings are, accordingly, to be regarded in an illustrative rather than restrictive sense. The claimed inventions are intended to cover alternatives, modifications, and equivalents.

What is claimed:

1. An apparatus for treating or preventing a kidney disease, comprising:
a delivery device configured for insertion into an urinary tract;
wherein the delivery device comprises a section for placement in the urinary tract; and
wherein the delivery device comprises a first end configured for coupling to an oxygen source, and a lumen configured for delivering oxygen from the oxygen source to a kidney.

2. The apparatus of claim 1, further comprising a controller configured for controlling oxygen flow from the oxygen source.

3. The apparatus of claim 2, further comprising a sensor for sensing a characteristic representative of an oxygen level, wherein the controller is configured for controlling the oxygen flow based at least in part on the sensed characteristic.

4. The apparatus of claim 3, wherein the controller is configured for controlling the oxygen flow based at least in part on the sensed characteristic to maintain a target oxygen level in urine.

5. The apparatus of claim 2, wherein the controller is configured for controlling a flowing rate of oxygen through the delivery device.

6. The apparatus of claim 2, wherein the controller comprises a user interface configured for receiving an input from a user to control a rate of oxygen flowing through the delivery device.

7. The apparatus of claim 6, wherein the user interface is configured for receiving an oxygen flow rate as the input.

8. The apparatus of claim 2, wherein the controller is configured to deliver oxygen continuously.

9. The apparatus of claim 2, wherein the controller is configured to deliver oxygen intermittently.

10. The apparatus of claim 1, further comprising an urinary catheter for draining urine from the kidney.

11. The apparatus of claim 1, further comprising an antibiotic medication for delivery by the delivery device.

12. The apparatus of claim 1, further comprising the oxygen source, wherein the oxygen source comprises fluid that is supersaturated with oxygen.

13. The apparatus of claim 1, further comprising a source of cooling material, wherein the delivery device is coupled to the source of cooling material, and is configured to deliver the cooling material to the kidney.

14. The apparatus of claim 13, wherein the cooling material comprises nitrous oxide, carbon dioxide, cooled fluid, liquid nitrogen, or argon gas.

15. The apparatus of claim 13, wherein the delivery device comprises an additional lumen for delivery of the cooling material.

16. The apparatus of claim 1, wherein the delivery device comprises a catheter.

17. A method for treating or preventing a kidney disease, comprising:
inserting a delivery device into an urinary tract;
advancing the delivery device distally inside the urinary tract until a distal end of the delivery device is at a desired position; and
delivering oxygen through the delivery device in the urinary tract to a kidney.

18. The method of claim 17, wherein the act of delivering oxygen comprises delivering oxygenated fluid.

19. The method of claim 18, wherein the oxygenated fluid is supersaturated with oxygen.

20. The method of claim 18, wherein the oxygenated fluid is delivered at a rate sufficient to reduce a kidney injury from a disease or from a surgery.

21. The method of claim 20, wherein the disease is sepsis, heart failure, infection, kidney inflammation, hypotension, or acute kidney failure.

22. The method of claim 17, further comprising delivering a cooling material through the urinary tract to cool the kidney.

23. The method of claim 22, wherein the kidney is cooled to prevent acute kidney injury.

24. The method of claim 22, wherein the kidney is cooled to inhibit inflammation of the kidney.

25. The method of claim 22, wherein the kidney is cooled to a temperature that is anywhere from 37 degree C. and −50 degree C.

26. The method of claim 22, wherein the kidney is cooled for a duration that is anywhere from 0.01 second to 8 weeks.

27. The method of claim 22, wherein the kidney is cooled continuously.

28. The method of claim 22, wherein the kidney is cooled intermittently.

29. An apparatus for treating or preventing a kidney disease, comprising:
a delivery device configured for insertion into an urinary tract;
wherein the delivery device comprises a section for placement in the urinary tract; and
wherein the delivery device comprises a first end configured for coupling to a source of cooling material, and a lumen configured for delivering the cooling material to a kidney.

30. The apparatus of claim 29, wherein the cooling material comprises nitrous oxide, carbon dioxide, cooled fluid, liquid nitrogen, or argon gas.

31. The apparatus of claim 29, wherein the delivery device is also configured for coupling to an oxygen source for delivering oxygen through the urinary tract to the kidney.

32. The apparatus of claim 31, wherein the delivery device comprises an additional lumen for delivery of the oxygen to the kidney.

33. The apparatus of claim 29, further comprising a controller configured for controlling flow of the cooling material.

34. The apparatus of claim 33, further comprising a sensor for sensing a characteristic representative of a temperature, wherein the controller is configured for controlling the flow of the cooling material based at least in part on the sensed characteristic.

35. The apparatus of claim 34, wherein the controller is configured for controlling the flow based at least in part on the sensed characteristic to maintain a target temperature.

36. The apparatus of claim 33, wherein the controller is configured for controlling a flowing rate of the cooling material through the delivery device.

37. The apparatus of claim 33, wherein the controller comprises a user interface configured for receiving an input from a user to control the flow of the cooling material.

38. The apparatus of claim 37, wherein the user interface is configured for receiving a temperature value as the input.

39. The apparatus of claim 29, further comprising an urinary catheter for draining urine from the kidney.

40. A method for treating or preventing a kidney disease, comprising:
inserting a delivery device into an urinary tract;
advancing the delivery device distally inside the urinary tract until a distal end of the delivery device is at a desired position; and
delivering cooling material through the delivery device in the urinary tract to a kidney.

41. The method of claim 40, wherein the kidney is cooled to prevent acute kidney injury.

42. The method of claim 40, wherein the kidney is cooled to inhibit inflammation of the kidney.

43. The method of claim 40, wherein the kidney is cooled to a temperature that is anywhere from 37 degree C. and −50 degree C.

44. The method of claim 40, wherein the kidney is cooled for a duration that is anywhere from 0.01 second to 8 weeks.
